(19) United States
(12) Patent Application Publication
(10) Pub. No.: US 2024/0173042 A1
(43) Pub. Date: May 30, 2024

(54) DEVICE FOR CLOT RETRIEVAL WITH VARYING TUBE DIAMETERS

(71) Applicant: Imperative Care, Inc., Campbell, CA (US)

(72) Inventors: Yi Yang, San Francisco, CA (US); Chad C. Roue, San Jose, CA (US); Miranda M. Ray, San Jose, CA (US)

(21) Appl. No.: 18/436,882

(22) Filed: Feb. 8, 2024

Related U.S. Application Data
(63) Continuation of application No. PCT/US2022/078113, filed on Oct. 14, 2022.
(60) Provisional application No. 63/256,743, filed on Oct. 18, 2021.

(51) Int. Cl. A61B 17/22; A61B 17/00; A61M 1/00

(52) U.S. Cl. CPC A61B 17/22; A61B 2017/00292; A61B 2017/22079; A61M 1/73; A61M 1/79; A61M 2205/3334

(57) ABSTRACT

An aspiration system may include an aspiration pump configured for location outside of a sterile field. The aspiration system may include a chamber configured for location within the sterile field. The aspiration system may include a first tube having a first inner diameter, the first tube configured to place the chamber in communication with an aspiration catheter. The aspiration system may include a second tube having a second inner diameter, the second tube extending between the chamber and the aspiration pump and configured to place the chamber and the aspiration pump in communication with each other, wherein the first inner diameter is larger than the second inner diameter.

### CROSS-REFERENCE TO RELATED APPLICATIONS

This application is a continuation application of International Patent Application No. PCT/US2022/078113 filed Oct. 14, 2022, which claims the priority benefit of U.S. Provisional Patent Application No. 63/256,743, filed Oct. 18, 2021, the entirety of each of which is hereby incorporated by reference herein.

### BACKGROUND

Thrombotic restrictions and occlusions within a patient's blood vessels are a significant medical problem and often require intervention to remove these restrictions and blockages to restore health to patients. While applicable to a wide range of vascular applications in both the arterial and venous systems, including a variety of small vessels such as arterial blockages in the neuro vasculature (ischemic stroke), the following background illuminates the problems primarily through the example of patients suffering with Pulmonary Embolisms.

Venous thromboembolic disease (VTE) is a worldwide crisis. There are over 10 million cases of deep vein thrombosis (DVT) and pulmonary embolism (PE) diagnosed globally per year, with 1 million cases occurring in the United States and over 700,000 in France, Italy, Germany, Spain, Sweden, and the United Kingdom combined each year. There are approximately 60,000 to 100,000 deaths from PE in the United States each year. DVT and PE are part of the same continuum of disease, with over 95% of emboli originating in the lower extremities. When PE occurs, the severity depends on the embolic burden and its effect on the right ventricle as well as underlying cardiopulmonary comorbidities. Death can result from the acute increase in pulmonary artery (PA) pressure with increased right ventricular (RV) afterload and dysfunction.

Patients with high-risk pulmonary embolism (PE) and other ischemic conditions were treated primarily with thrombolytic therapy delivered systemically or more locally through Catheter Directed Thrombolytics. These approaches result in multiple catheterization lab visits, lengthy hospital stays and often lead to bleeding complications. Newer approaches to both PE and ischemic stroke include single session thrombectomy (aspiration of the clot) without the use of thrombolytics. These thrombectomy treatments include positioning the distal end of an aspiration catheter adjacent the clot and applying suction in an effort to aspirate the clot through the catheter and into a canister outside of the sterile field.

One challenge for the physician is knowing when a clot has been successfully captured, as well as the volume of the clot, to inform next steps and allow blood loss to be minimized. Although it is theoretically possible to monitor clot accumulation in the canister associated with the aspiration pump, the practical setup in the current cath lab environment makes it difficult for the physician to obtain easy access to this information.

### SUMMARY

Disclosed is a vacuum aspiration system, such as for aspirating a target material such as an obstruction from the vascular system. In particular, a clot retrieval device is provided for use in-line with the aspiration system to capture and filter out the targeted clot. The clot retrieval device can allow capture and visualization of the clot at a location near or on the aspiration catheter hub, within the sterile field and spaced apart from the vacuum canister. The clot retrieval device can optionally include a valve to generate pulsations in vacuum pressure. A pulsatile pump can help to improve the aspiration of the clot by “shaking” the clot loose.

In some embodiments, disclosed is a clot filtering device comprising a body defining a cavity and having a filter in the cavity. In some embodiments, the body comprises a first port on a first side of the filter, and a second port on a second side of the filter in fluid communication with the first port through the filter. In some embodiments, the filter comprises a plurality of openings in communication with the cavity.

In some embodiments, the body comprises a tubular sidewall such as a cylindrical side wall having a longitudinal axis. In some embodiments, an upstream surface of the filter may reside on a plane that resides at a non normal angle to the longitudinal axis. The inclined angle of the filter allows elongation of a major axis of the filter into an elliptical shape within the body, increasing the surface area of the filter configured to interact with aspirated material passing through the body. In some embodiments, the filter is angled between 30 to 90 degrees with respect to the longitudinal axis.

In some embodiments, each of the plurality of openings of the filter is less than or equal to 1 mm. In some embodiments, the plurality of openings of the filter is configured to prevent a clot from passing through the filter. In some embodiments, the first port and the second port are positioned on opposite ends of the body. In some embodiments, the body further comprises a third port, wherein the third port is a flush port configured to allow the injection of saline or other fluid into the body of the clot filtering device.

In some embodiments, the body is at least partially transparent. In some embodiments, the body comprises a top portion and a bottom portion enclosing a cavity, and wherein the filter is positioned within the cavity between the top portion and the bottom portion. In some embodiments, a valve is configured vent to atmosphere or to a second vacuum source. In some embodiments, the device further includes a button, wherein the button can allow a user to manually actuate the valve. In some embodiments, the button is positioned on a top portion of the body.

In some embodiments, the device further includes a sensor configured determine when flow has stopped in the clot filtering device. In some embodiments, the valve is automatically engaged when the sensor has determined that flow has stopped. In some embodiments, the first port is positioned on a proximal end of the top portion and the bottom portion, and wherein the second port is positioned on a distal end of the top portion or the bottom portion.

In some embodiments disclosed is a system for clot aspiration including an aspiration catheter, a first catheter, a clot filtering device, a second catheter, and a pump. A length of tubing between the clot filtering device and the catheter is significantly less than a length of tubing between the clot filtering device and the pump. In some embodiments, the clot filtering device includes a body and a filter. In some embodiments, the body includes a first port and a second port, wherein the body comprises a circular cross-section. In some embodiments, the filter includes a plurality of openings, the filter positioned in the body of the clot filtering device. In some embodiments, the first catheter is fluidly connected to the aspiration catheter and the clot filtering device. In some embodiments, the second catheter is fluidly connected to the pump and the clot filtering device.

In some embodiments, the system further comprises a clamp disposed over the first catheter, wherein the clamp can be engaged reduce flow through the clot filtering device. In some embodiments, the system includes a body that is cylindrical. In some embodiments, the system includes a clot filtering device with a filter that is angled within the body to increase the surface area configured to interact with aspirated material passing through the body. In some embodiments, the system includes a clot filtering device includes a filter that is angled between 30 to 90 degrees with respect to a flow axis within the body. In some embodiments, the system includes a clot filtering device wherein the filter includes a plurality of openings that is less than or equal to 1 mm. In some embodiments, the system includes a clot filtering device wherein the filter includes a plurality of openings that is configured to prevent a clot from passing through the filter. In some embodiments, the first port and the second port of the clot filtering device are positioned on opposite ends of the body.

In some embodiments, the body of the clot filtering device includes a third port, wherein the third port is a flush port configured to allow the injection of saline or other fluid into the body of the clot filtering device. In some embodiments, the body of the clot filtering device is at least partially transparent. In some embodiments, the body of the clot filtering device comprises a top portion and a bottom portion, wherein the filter is positioned between the top portion and the bottom portion. In some embodiments, the valve of the clot filtering device is configured vent to a second vacuum source.

In some embodiments, the clot filtering device includes a button, wherein the button can allow a user to manually actuate the valve. In some embodiments, the button of the clot filtering device is positioned on a top portion of the body.

In some embodiments, the clot filtering device further comprises a sensor configured determine when flow has stopped in the clot filtering device. In some embodiments, the valve of the clot filtering device is automatically engaged when the sensor has determined that flow has stopped. In some embodiments, in the clot filtering device, the first port is positioned on a proximal end of the top portion and the second portion, and the second port is positioned on a distal end of the portion and the second portion.

In some embodiments, disclosed is a clot filtering device. In some embodiments, the clot filtering device comprises a cylindrical body comprising a first port and a second port positioned on opposite ends of the cylindrical body. The body may comprise a circular cross-section and at least a portion of the sidewall the cylindrical body is at least partially optically transparent. In some embodiments, the clot filtering device comprises a filter comprising a plurality of openings, the filter positioned in the body of the clot filtering device, the filter angled within the body to increase the surface area configured to interact with aspirated material passing through the body wherein the filter is angled between 30 to 90 degrees with respect to an inner circumference of the body.

In some embodiments, disclosed is a clot filtering device. In some embodiments, the clot filtering device comprises a body comprising a first port and a second port positioned on opposite ends of the body, wherein the body comprises a circular cross-section and is at least partially transparent. In some embodiments, the clot filtering device comprises a filter comprising a plurality of openings, the filter positioned in the body of the clot filtering device. In some embodiments, the clot filtering device comprises a valve configured vent to a second vacuum source. In some embodiments, the clot filtering device comprises a button configured to allow a user to manually actuate the valve, the button positioned on a top portion of the body. In some embodiments, in the clot filtering device, the body comprises a top portion and a bottom portion, wherein the filter is positioned between the top portion and the bottom portion. In some embodiments, in the clot filtering device, the first port is positioned on a proximal end of the top portion and the second portion, and the second port is positioned on a distal end of the portion and the second portion.

### BRIEF DESCRIPTION OF THE DRAWINGS

These and other features, aspects and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters denote corresponding features consistently throughout similar embodiments. The following is a brief description of each of the drawings.

FIG. 1 is a schematic of a fluid management system for aspiration procedures.

FIG. 2 illustrates an embodiment of a schematic of a fluid management system for aspiration procedures according to the schematic of FIG. 1.

FIGS. 3A-3C illustrates an embodiment of an inline clot retrieval device.

FIGS. 4A-4C illustrates another embodiment of an inline clot retrieval device.

FIGS. 5A-5B illustrates another embodiment of an inline clot retrieval device.

FIG. 6A illustrates a front perspective view of an embodiment of an inline clot retrieval device.

FIG. 6B illustrates a cross-sectional perspective view of the embodiment of FIG. 6A.

FIG. 6C illustrates a rear perspective view of the embodiment of FIG. 6A.

FIGS. 6D and 6E illustrates a front and rear cross-sectional view of the embodiment of FIG. 6A.

FIGS. 6F-6H illustrate side views of the embodiment of FIG. 6A.

FIGS. 6I-6K illustrates side view of the embodiment of FIG. 6A.

FIG. 7 is another schematic view of a fluid management system for aspiration procedures.

FIGS. 8A-8B illustrate front and rear views of another embodiment of an inline clot retrieval device.

FIGS. 8C-8D illustrate front and rear cross-sectional view of the embodiment of FIGS. 8A-8B.

FIGS. 9A-9B illustrate the embodiment of the inline clot retrieval device of FIGS. 8A-8D in use.

FIGS. 10A-10F illustrate an embodiment of an inline clot retrieval device.

### DETAILED DESCRIPTION

### Overview

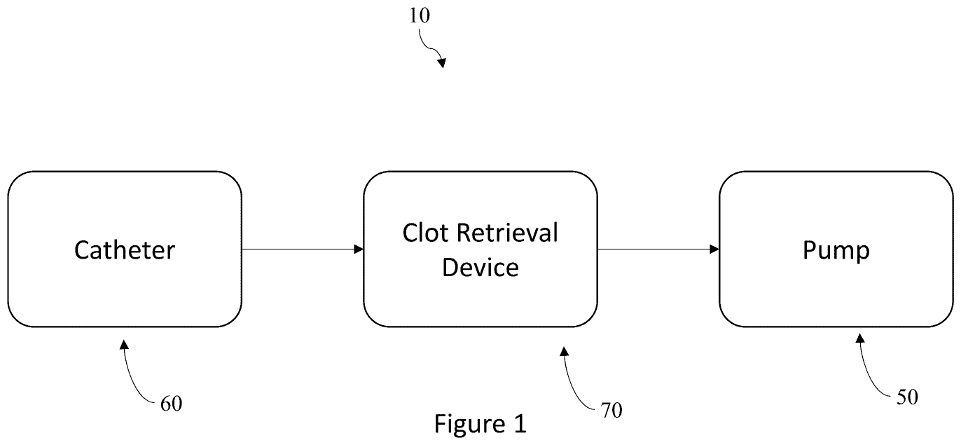

FIG. 1 illustrates a schematic of a fluid management system for aspiration procedures. The fluid management system 10 can include a catheter 60, a clot retrieval device 70, and an aspiration pump 50. The catheter 60, clot retrieval device 70, and the aspiration pump 50 can be fluidly connected such that fluid is aspirated from a distal end of the catheter 60 and into and through the clot retrieval device 70.

The length of tubing between the clot retrieval device 70 and the pump 50 may be substantially greater than the length of tubing between the clot retrieval device 70 and the catheter 60. This enables the clot retrieval device 70 to be positioned with in the sterile field for easy direct visualization by the physician during the aspiration procedure, while the pump 50 may remain relatively remote from the physician and outside of the sterile field. Additional details of sterile field clot retrieval devices and associated fluidics may be seen in U.S. patent application Ser. No. 17/357,558, filed Jun. 24, 2021 and entitled Aspiration System with Accelerated Response, the disclosure of which is hereby expressly incorporated in its entirety herein by reference.

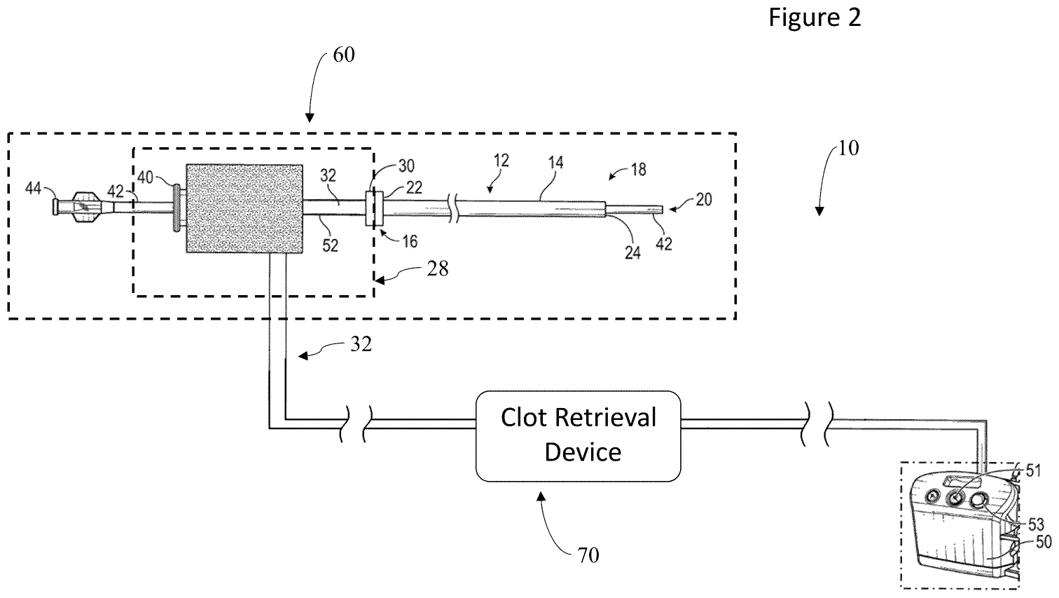

FIG. 2 illustrates an embodiment of a catheter 60 that can be used with the system 10. As shown, the system 10 can include a large diameter first thrombectomy catheter 12, having an elongate tubular body 14 extending between the proximal end 16 and a distal end 18. A central lumen 20 extends between a proximal catheter connector 22 and a distal port 24 on the distal end 18.

In the illustrated embodiment, the catheter 12 can be releasably connectable to a flow control module 28 by way of a complementary module connector 30. Module connector 30 provides a releasable connection to complementary catheter connector 22 and may include an opener (not illustrated) for opening a hemostasis valve in the hub of the large bore catheter (not illustrated).

In some embodiments, the flow control module 28 can include a fluid flow path 32 extending between the module connector 30 and the flow control module 28. The fluid flow path 32 continues to extend between the flow control module 28 and the clot retrieval device 70, which contains a filter for thrombus collection and/or evaluation and a chamber for filtered fluid chamber (discussed in more detail below). In some examples, the flow control module 28 is integrally formed within the hub of thrombectomy catheter 12 to which the catheter may be non-removably attached. In addition, the flow path between the flow control module 28 and the clot retrieval device 70 may be contained within a continuous integral tubing or may be contained within two or more tubing components releasably connectable via complementary Luer locks or other connectors.

The flow control module 28 can include a flow regulator for regulating flow through the flow path 32. The flow regulator can provide a reversible restriction in the flow path, such as by an expandable or contractible iris, a ball valve or other rotary core valve, leaf valve, a pinch tubing, or others known in the art.

In some embodiments, the flow regulator comprises a collapsible portion of the tubular wall defining the flow path, such as a section of polymeric tubing. An actuator positioned adjacent the tubing is movable between a first position where it compresses the tubing, thereby restricting flow to the low flow rate, and a second position where it has moved away from the tubing, allowing the tubing to resume its full inside diameter and allow the high flow rate. The actuator may be spring biased or have other default driver in the direction of the first (restricted) position, and only movable into the second position in the presence of an affirmative mechanical force or electrical signal that actuates the high flow override. Upon removal of the momentary override command, the actuator automatically resumes the first, position, producing the low flow mode.

The actuator may be driven by a mechanical control such as a lever or rotatable knob, or an electrically driven system such as a solenoid, operated by any of a variety of buttons, levers, triggers, foot pedals or other switches known in the art, depending upon the desired functionality.

In some embodiments, the fluid flow may be selectively directed through a low flow regulator such as a small diameter orifice or tube, and a high flow regulator such as a larger diameter orifice or tube. A mechanically actuated or electromechanically actuated valve can momentarily divert flow from the low flow to the high flow regulator in response to actuating a control.

Flow control module 28 thus includes one or more controls, for controlling the operation of the system. One control may be provided for toggling the system between a no flow (off) mode and a low flow mode. In some embodiments, the same or a different control may be provided for momentarily toggling the flow regulator between the low flow mode and a momentary operator initiated high flow override mode. Release of the momentary override control causes the regulator to revert to off or low flow mode.

In some examples, the low flow mode enables the first catheter 12 to approach and engage the clot with a relatively low volume of blood aspiration. Once the clot is engaged, the momentary high flow control may be activated to generate a bolus of high flow vacuum to draw the clot into the catheter 12. High flow may be at least about 10 cc/second, and preferably at least about 15 cc/sec but typically no more than about 25 cc/sec. In one construction the high flow rate is about 20 cc/sec, with all of the foregoing flow rates in an unobstructed aspiration of blood. Low flow as used herein is no more than about 50%, no more than about 35% or no more than about 25% of the high flow rate. Low flow is generally less than about 10 cc/sec or 7 cc/sec, and is often in the range of from about 1-5 cc/sec.

The flow control module 28 may be provided with a second catheter port 40 in communication with central lumen 20 via a hemostasis valve (e.g., Tuohy Borst valve) (not illustrated) within the module 28. This allows introduction of a second aspiration catheter 42 through the access catheter 12 and extending to the treatment site. The second catheter 42 may be a smaller diameter aspiration catheter, with or without clot agitation or mechanical grasping capabilities, drug delivery catheter, a mechanical disrupter or other accessory device that may be useful in the clot retrieval process. In one implementation, the second catheter including its hand piece and controls may be identical in material respects to the first aspiration catheter except the second catheter is smaller diameter and longer than the first catheter.

If desired, the second catheter 42 may be connected via a proximal connector 44 to a complementary connector 46 which is in communication with a reservoir (not illustrated) via an additional aspiration line. In some embodiments, the second aspiration catheter 42 may be connected to the clot retrieval device 70.

The clot may be removable through the first catheter 12 under vacuum without additional assistance. However, if desired, the secondary clot grasping catheter 42 may be introduced to provide additional attachment and/or mechanical disruption of the clot to facilitate removal. Removal may be assisted by the application of vacuum to the grasping catheter 42 as well as to the first catheter 12 in sequence or simultaneously depending upon the desired clinical performance.

As shown in FIG. 2, the aspiration pump 50 can be fluidly connected to the catheter 60. The aspiration pump 50 can include a vacuum pump, and may also include a vacuum gauge 51, and an optional a pressure adjustment control 53. In some embodiments, the vacuum gauge 51 is in fluid communication with the vacuum pump and provides an indication of the vacuum pressure generated by the pump. The pressure adjustment control 53 allows the user to set to a specific vacuum pressure. Any of a variety of controls may be utilized, including switches, buttons, levers, rotatable knobs, and others which will be apparent to those of skill in the art in view of the disclosure herein. In some examples, the aspiration pump 50 may be a manually activated pump such as a syringe.

In some embodiments, the system 10 can include a reservoir (not illustrated) that is in fluid communication with the aspiration pump 50 via vacuum line 35 and acts to transfer vacuum from the air-filled side of the system to the liquid side of the system and to collect aspirated filtered blood. In some examples, the reservoir includes a collection canister in fluid communication with flow path 32 and collects aspirated debris. In some embodiments, the flow direction through the system may also be reversed to allow the blood to flow through the filter while the clot is collected outside (now downstream) of the filter, e.g., between the filter and the outer transparent window or container. The reservoir may be located “upstream” or proximal to the clot retrieval device 70 such that the reservoir is in fluid communication between the clot retrieval device 70 and the pump 50.

In some examples, the flow path 32 extends throughout the length of the first catheter 12, through the control module 28 and into the reservoir 34. The flow path 32 can include a transparent window 52 to enable direct visualization of the contents of the flow path 32. In some embodiments, the window 52 is in the form of a transparent section of tubing between the proximal end of the catheter 12 and the flow module 28, and within the sterile field so that the clinician can directly visualize debris as it exits the proximal end of the access catheter 12. In some examples, the length of the transparent tubing can be at least about two or four or 6 cm long and generally less than about 30 or 20 cm long. In some embodiments, the length of the transparent tube is within the range of about 5 cm to about 15 cm. In some examples, the transparent window may be carried by the proximal hub of the catheter 12 or may be a proximal portion of the catheter shaft, distally of the hub.

In some embodiments, the system 10 can include a clot retrieval device 70. The clot retrieval device 70 can be fluidly connected to the catheter 60 and the aspiration pump 50. As will be described in more detail below, the clot retrieval device 70 can be handheld within the sterile field and allow clinician to capture and visualize the clot during the aspiration procedure. The clot retrieval device 70 can include a filter that catches solid clot debris as fluid is aspirated through the catheter 60. The clot retrieval device 70 can include an additional port for injecting saline or other fluid into the clot retrieval device 70 to improve clot visualization once it is trapped in the filter.

In some embodiments, the clot retrieval device 70 can include a valve that allows a pulsation of the pressure being applied by the aspiration pump 50. For example, the clot retrieval device 70 can include a button or other actuator that can be engaged by the user to close the valve in the clot retrieval device 70 and reduce the amount of pressure applied by the aspiration pump 50. The clinician can thereafter release the button and allow pressure to be reapplied.

### Clot Retrieval Device

As shown in FIG. 1, in some embodiments, the system 10 can include a clot retrieval device 70. The clot retrieval device 70 can be configured to capture a clot that is removed by the system 10 during an aspiration procedure performed in a patient's clogged vessel. As will be discussed, in more detail below, the clot retrieval device 70 can be an in-line canister that is positioned between a proximal end of an aspiration catheter (e.g., catheter 60) and an aspiration source (e.g., aspiration pump 50).

In some embodiments, the length of the tubing between the clot retrieval device 70 and the catheter 60 is no more than about 50% or 25% or 15% or less than the length of the tubing between the clot retrieval device 70 and the pump 50, such that the clot retrieval device 70 can be within the sterile field and easily directly viewed by the physician holding the aspiration catheter manifold, while the pump 70 can be remotely located outside of the sterile field.

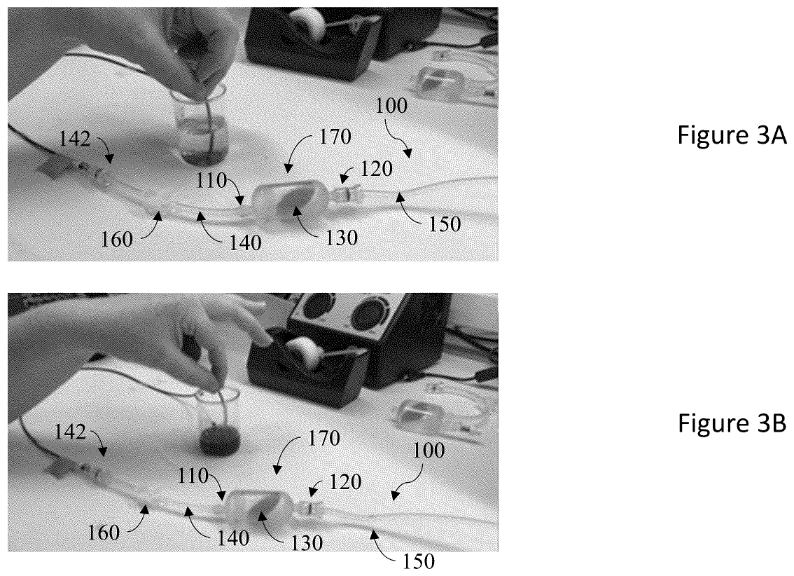

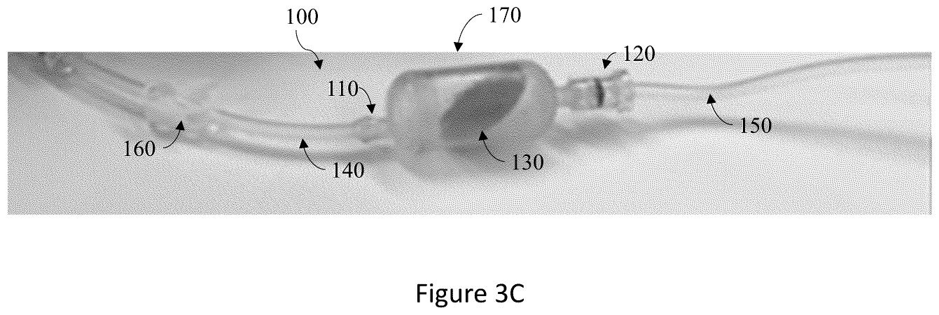

FIGS. 3A-3C illustrate a system 100 with an embodiment of a clot retrieval device 170. In some embodiments, the clot retrieval device 170 includes a first port 110, a second port 120, and a filter 130. In some examples, the first port 110 is configured to connect to a first end of a first tube 140 that is fluidly connected to a proximal end of the aspiration catheter. In some embodiments, the first tube 140 includes a connector 142 positioned at a second end of the first tube 140 that is configured to engage or mate with a corresponding connector.

In some embodiments, the second port 120 is configured to connect to a first end of a second tube 150 that is fluidly connected to an aspiration source (e.g., a pump). In some embodiments, the second tube 150 includes a connector (not illustrated) positioned at a second end of the second tube 150 that is configured to engage or mate with a corresponding connector. In some examples, the system 100 can include a clamp 160. In some embodiments, the clamp 160 can be positioned over the first tube 140 to allow the user to engage the clamp and provide flow control over the clot retrieval device 170.

In some embodiments, the body of the clot retrieval device 170 can include a tubular side wall such as a generally cylindrical sidewall with a longitudinal axis and a circular transverse cross-section defining an interior chamber. The clot retrieval device 170 can be made of any of a variety of materials known in the art, including optically transparent polymers. As illustrated in FIGS. 3A-3C, in some embodiments, at least a window portion of the clot retrieval device 170 can be optically transparent to improve clot visualization once it is trapped in the clot retrieval device 170.

The clot retrieval device 170 may include a filter 130 within the chamber. In some embodiments, the filter 130 can be shaped to divide the chamber into an upstream side and a downstream side. In some examples, at least an upstream surface of the filter 130 can be angled to increase the surface area that can interact with the aspirated clot. For example, the upstream surface of the filter 130 can lie on a plane that is inclined within the range of from about 30 to 90 degrees with respect to the longitudinal axis of the chamber. In some embodiments, the filter 130 can be angled in the clot retrieval device 170 at an angle of at least about 30 degrees, at least about 35 degrees, at least about 40 degrees, or at least about 50 degrees, and generally no more than about 70 degrees, or 80 degrees from the longitudinal axis.

In some examples, the filter 130 may be removable from the canister of the clot retrieval device 170 such that the filter 130 may be cleaned, replaced or adjusted. In some examples, filters may be provided having different pore sizes, that may be selected depending upon the desired performance. For example, in some embodiments, the filter 130 can include approximately 1 mm holes that catches larger solid clot debris as it is driven through the mini canister. In some embodiments, the pore sizes may have a maximum cross sectional dimension of no more than about 1 mm, no more than about 2 mm or no more than about 3 mm or 4 mm.

In some examples, aspirated material is drawn through the tube 140 and enters the first catheter port 110 of the clot retrieval device 170. The aspirated material can be filtered by the filter 130 of the clot retrieval device 170. Material that is greater than the openings on the filter 130 can be prevented from leaving the clot retrieval device 170. In some embodiments, blood and any material that is smaller than the openings on the filter 130 flows out of the second port 120 and the second tube 150, on to a collection canister associated with the pump.

In some examples, the clinician can slow down the flow of aspirated material by engaging the clamp 160. In some embodiments, the clinician can inspect the clot or other larger aspirated material caught by the filter 130. In some embodiments, the material retained by the clot retrieval device 170 can be removed from the clot retrieval device 170 for evaluation and/or disposal.

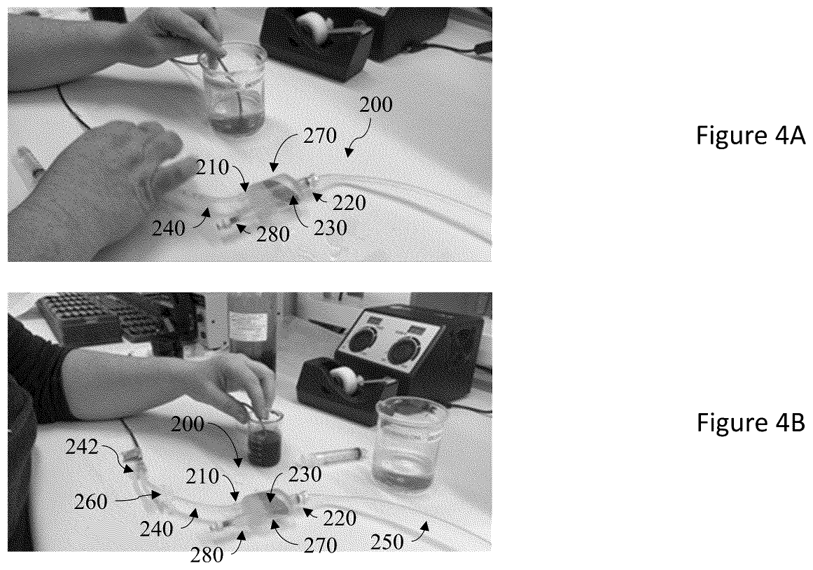

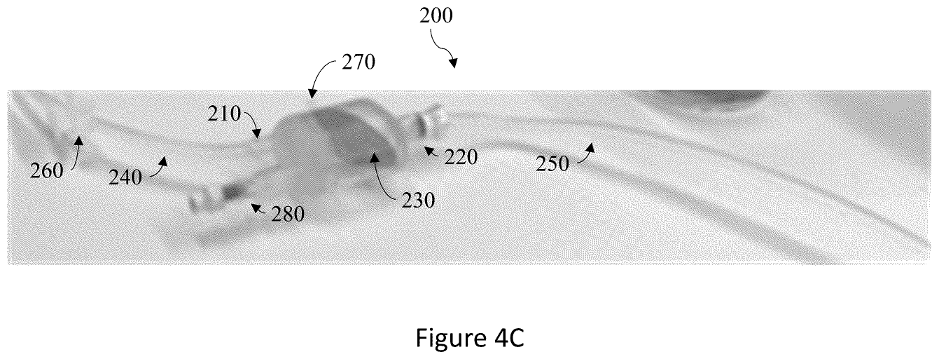

FIGS. 4A-4C illustrate a system 200 with another embodiment of a clot retrieval device 270. FIGS. 4A-4C resemble or are identical to the system 200 in many respects. Accordingly, numerals used to identify components of the system 200 are incremented by a factor of one hundred to identify like features of the system 200. This numbering convention generally applies to the remainder of the figures. Any component or step disclosed in any embodiment in this specification can be used in other embodiments. As with the clot retrieval device 170, the clot retrieval device 270 can include a first port 210, a second port 220, a third port 280, and a filter 230.

In some embodiments, the first port 210 is configured to connect to a first end of a first tube 240 that is fluidly connected to a proximal end of the aspiration catheter. In some examples, the first tube 240 includes a connector 242 positioned at a second end of the first tube 240 that is configured to engage or mate with a corresponding connector. In some embodiments, the second port 220 is configured to connect to a first end of a second tube 250 that is fluidly connected to an aspiration source (e.g., at pump). In some examples, the second tube 250 includes a connector (not illustrated) positioned at a second end of the second tube 250 that is configured to engage or mate with a corresponding connector.

In some embodiments, the clot retrieval device 270 includes the third port 280 for injecting saline or other fluid into the canister of the clot retrieval device 270. In some examples, this can improve clot visualization once it is trapped in the filter 230. In some embodiments, the system 200 can include a clamp 260. The clamp 260 can be positioned over the first tube 240 to allow the user to engage the clamp and provide flow control over the clot retrieval device 270.

In some examples, the body of the clot retrieval device 270 can include a tubular sidewall enclosing a cavity, such as a cylindrical side wall having a longitudinal axis and a circular transverse cross-section. In some embodiments, one or more components of the clot retrieval device 270 can be made of an optically transparent material, to improve clot visualization once it is trapped in the clot retrieval device 270.

Similar to the clot retrieval device 170, the clot retrieval device 270 can include a filter 230. In some embodiments, the filter 230 can be circular in shape, and oriented transverse to the longitudinal axis and the axis of fluid flow. In some examples, this can allow the filter 230 to rotate in the body of the clot retrieval device 270. In some embodiments, the filter 230 can be inclined at a non normal angle with respect to the longitudinal axis to increase the surface area that can interact with the aspirated clot.

For example, the filter 230 can lie on a plane that is inclined with respect to the longitudinal axis at an angle within the range of from about 30 to about 90 degrees. In some embodiments, the filter 230 can be angled in the clot retrieval device 270 at an angle of at least about 30 degrees, at least about 40 degrees, at least about 45 degrees or 55 degrees or more but generally no more than about 85 degrees or 75 degrees or 60 degrees with respect to the longitudinal axis

In some examples, the filter 230 may be removable from the body of the clot retrieval device 270 such that the filter 230 may be replaced, inspected or adjusted. In some embodiments, the clot retrieval device 270 may be provided with a variety of filters having different pore sizes based on the desired clinical performance. For example, in some embodiments, the filter 130 can include 1 mm holes that catches solid clot debris as it is driven through the mini canister. In some embodiments, the pore sizes may be at most 1 mm, at most 2 mm, at most 3 mm, at most 4 mm, at most 5 mm.

In some embodiments, the aspiration catheter of the system 200 aspirates material that flows through the second end of the first catheter 240 and enters the first catheter port 210 of the clot retrieval device 270. The aspirated material can be filtered by the filter 230 of the clot retrieval device 270. Material that is greater than the openings on the filter 230 can be prevented from leaving the clot retrieval device clot retrieval device 270. In some embodiments, material that is smaller than the openings on the filter 230 flows out of the second catheter port 220 and the second catheter 250. In some examples, the user can inspect the clot or other larger aspirated material caught by the filter 230. In some embodiments, the material retained by the clot retrieval device 270 can be removed from the clot retrieval device 270 and tested by the user.

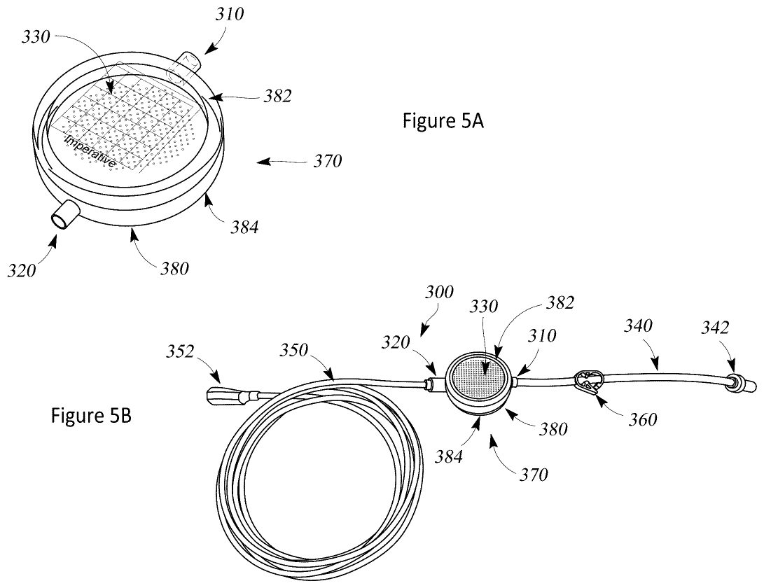

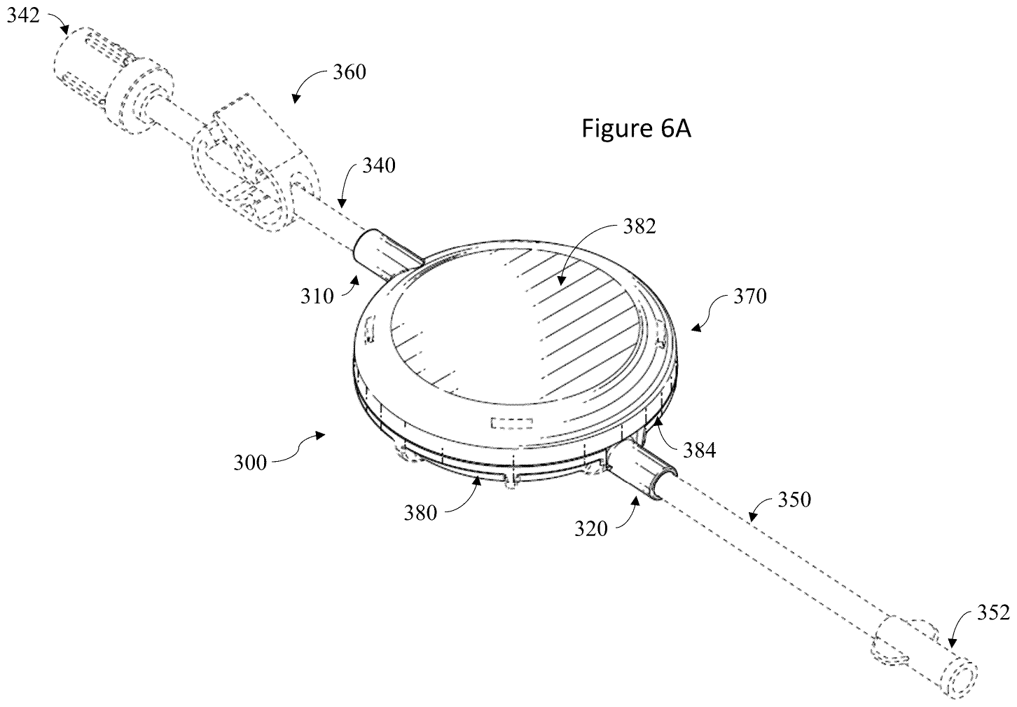

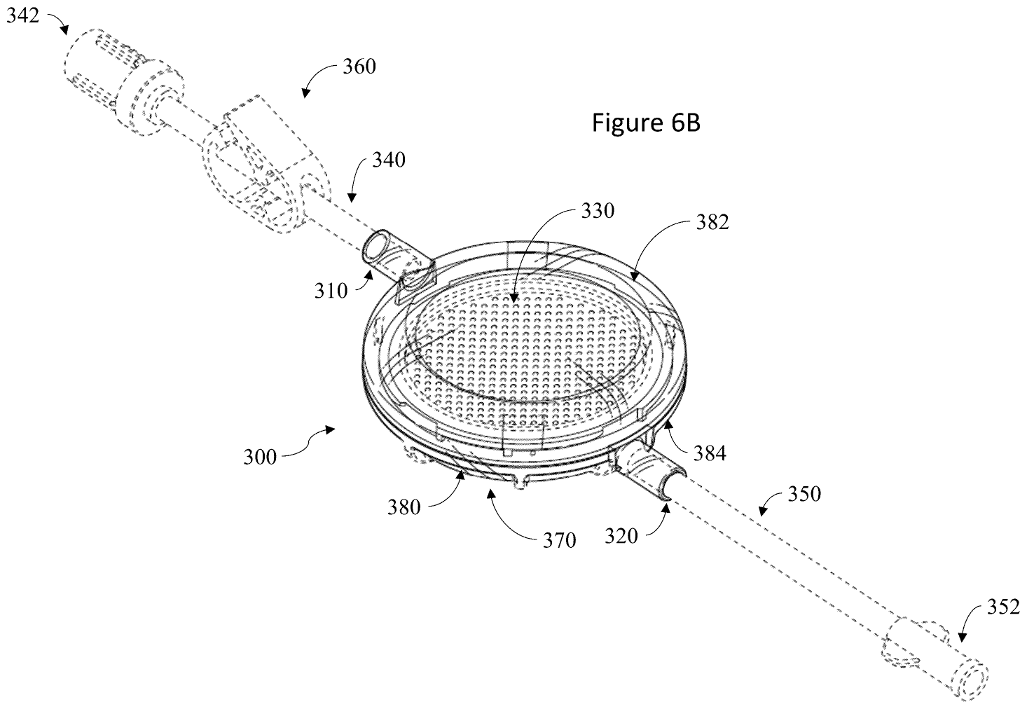

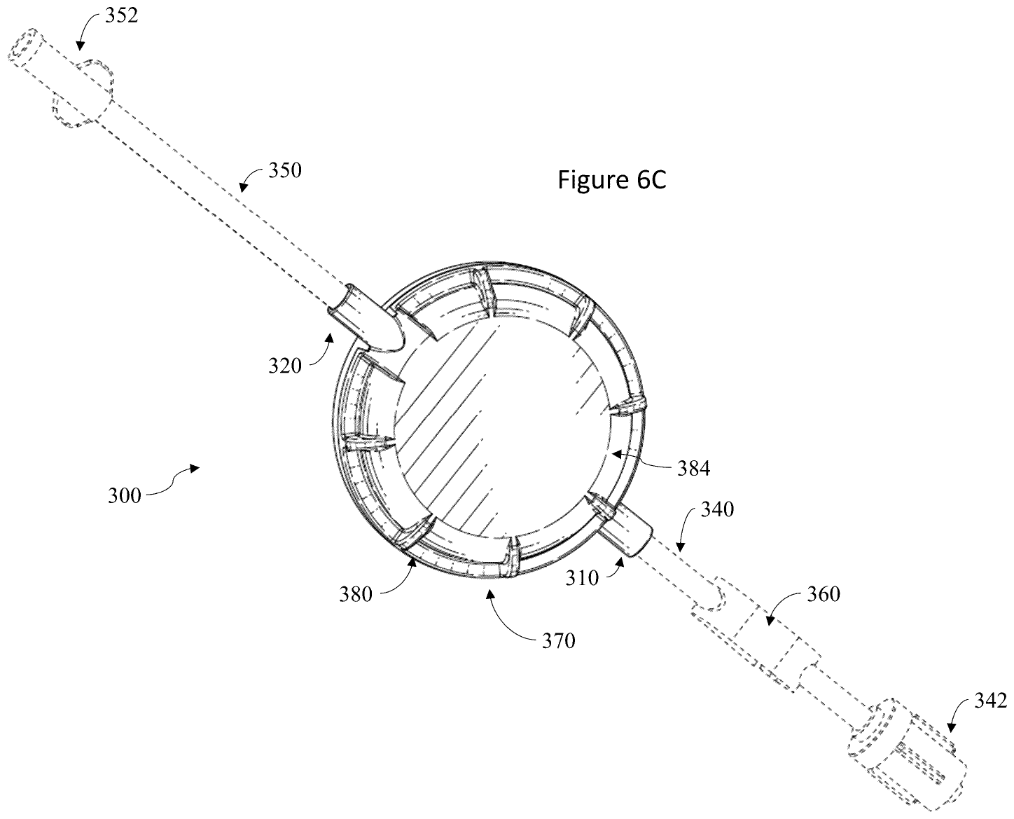

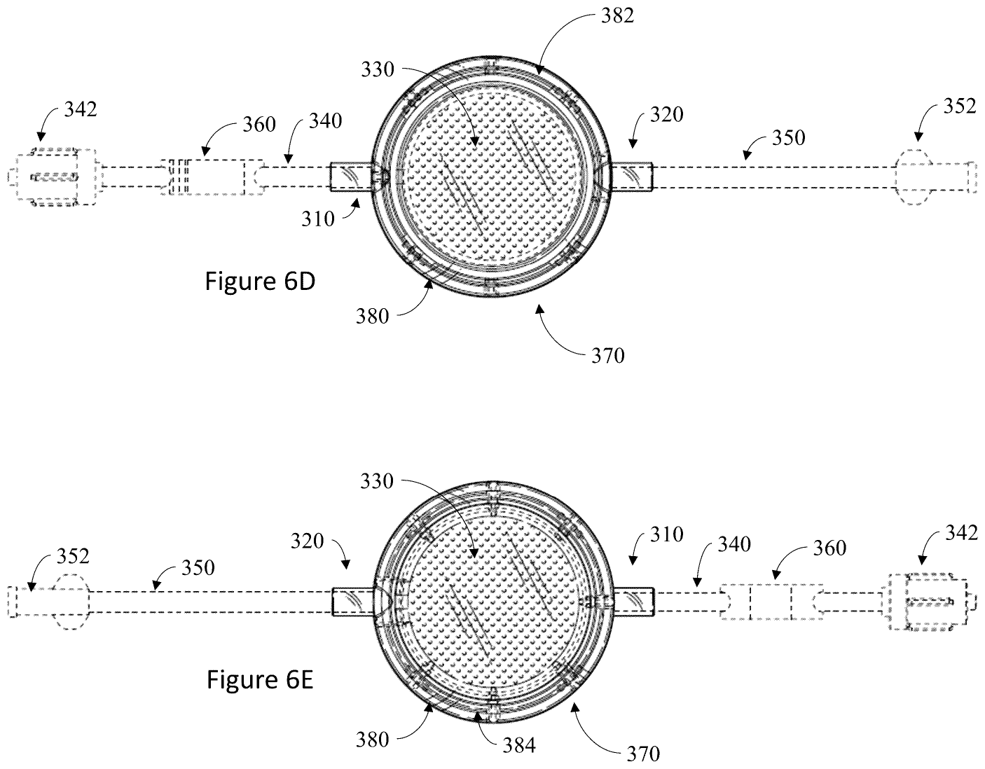

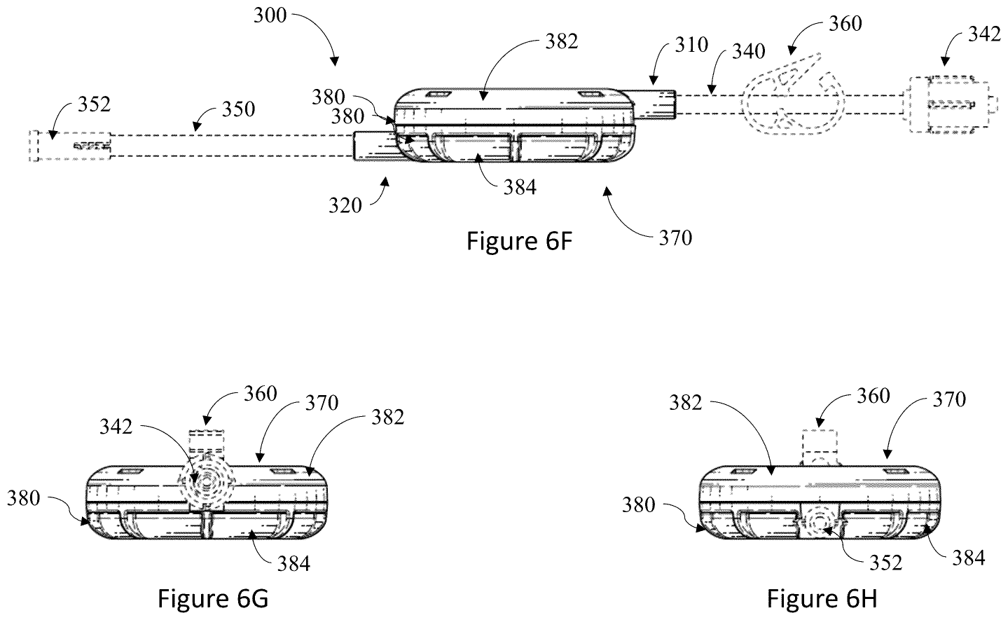

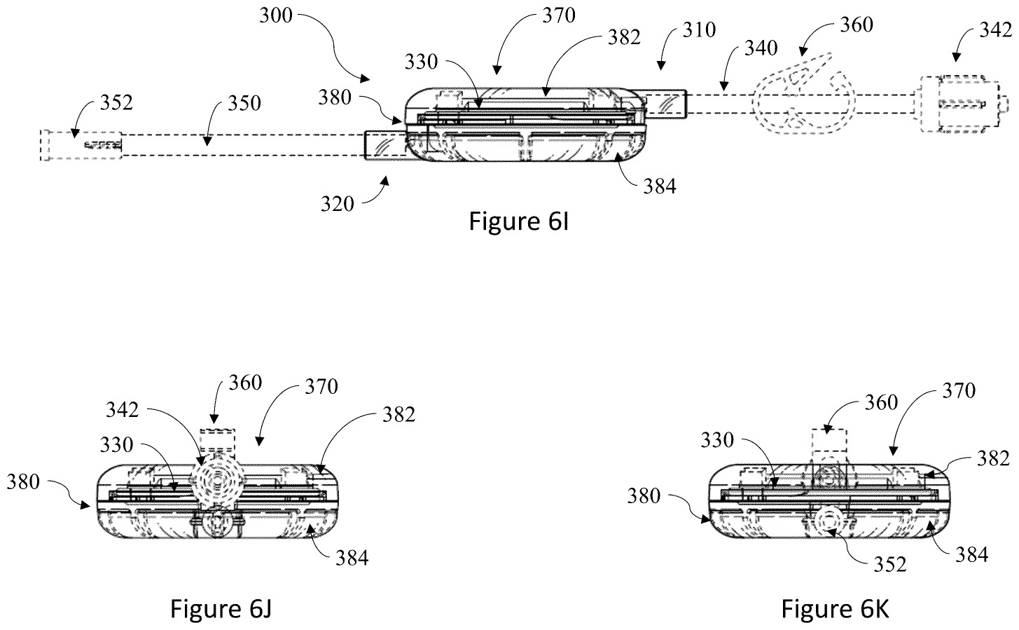

FIGS. 5A-5B and 6A-6K illustrates another embodiment of a clot retrieval device 370. The clot retrieval device 370 can include a body 380 enclosing a chamber which communicates with a first port 310 and a second port 320. In some examples, the body 380 can include a flush port (not illustrated) that is configured to allow the injection of saline or other fluid into the chamber to improve clot visualization once it is trapped in the filter 330.

In some embodiments, the body 380 includes a housing having a top portion 382 and a bottom portion 384. In some examples, the body 380 includes a filter 330 positioned in the chamber between the top portion 382, and the bottom portion 384. In some examples, the first port 310 is configured to connect to a first end of a first tube 340 that is fluidly connected to a proximal end of an aspiration catheter. In some embodiments, the first tube 340 includes a connector 342 positioned at a second end of the first tube 340 that is configured to engage or mate with a corresponding connector.

In some embodiments, the second port 320 is configured to connect to a first end of a second tube 350 that is fluidly connected to an aspiration source (e.g., a pump). In some embodiments, the second tube 350 includes a connector 352 positioned at a second end of the second tube 350 that is configured to engage or mate with a corresponding connector. In some examples, the system 300 can include a clamp 360. The clamp 360 can be positioned over the first tube 340 to allow the user to engage the clamp and provide flow control over the clot retrieval device 370.

As shown in the Figures, the housing of the clot retrieval device 370 can have a top surface spaced apart from a bottom surface by a tubular side wall. In the illustrated implementation, the top and bottom surfaces are substantially circular, and spaced apart by a cylindrical side wall having a diameter that is at least about three times, or five times or more than the axial length of the side wall, to produce a generally disc shaped housing. Preferably at least a portion of the top wall is optically transparent to improve clot visualization once it is trapped in the clot retrieval device 370. As shown in FIGS. 6B, 6D-6E, and 6I-6K, in some embodiments, at least one or both of the top portion 382 and the bottom portion 384 is transparent such that the filter 330 is visible through the body of the clot retrieval device 370.

In some embodiments, the clot retrieval device 370 can include a filter 330. The filter 330 can be circular in shape. In some embodiments, the filter 330 can be secured between the top portion 382 and the bottom portion 384 of the body 380. In some examples, the clot retrieval device 370 may be provided with different pore sizes based on the needs of the physician or the parent. For example, in some embodiments, the filter 330 can include 1 mm holes that catches solid clot debris as it is driven through the mini canister. In some embodiments, the pore sizes may be at most 1 mm, at most 2 mm, at most 3 mm, at most 4 mm, at most 5 mm.

In some embodiments, the aspiration catheter aspirates material which can flow through the second end of the first catheter 340 and enters the first catheter port 310 of the clot retrieval device 370. The aspirated material can then be filtered by the filter 330. Material that is greater than the openings on the filter 330 is prevented from leaving the clot retrieval device 370, while material that is smaller than the openings on the filter 330 flows out of the second catheter port 320 and the second catheter 350. In some embodiments, the user can slow down flow by engaging the clamp 360. In some examples, the user can inspect the clot or other larger aspirated material caught by the filter 330. In some embodiments, the material retained by the clot retrieval device 370 can be removed from the clot retrieval device 370 and tested by the user.

Clot Retrieval Device with Pulsatile Pump

In some examples, the clot retrieval device can include a pulsatile pump. The pump can be automatic or manual to allow the user to manipulate a control such as a valve located within the clot retrieval device. The pulsatile pump can allow the clot retrieval device to improve the ingestion of clot during the clinical procedure. This can serve as a simple and cost-effective manual vacuum on the clot retrieval device that is near or at the hub of the catheter instead of at the canister/pump. In some embodiments, the valve can also function as a clearing mechanism to allow the physician to visualize the clot during the procedure.

In some embodiments, the valve added to the clot retrieval device can momentarily release the vacuum and therefore create a pulse in the vacuum pressure. The venting can be to a second vacuum source (e.g., not to air) such that the device will not experience a decay in pressure, and it will never go positive. The second vacuum source can produce a second negative pressure that is less than a first negative pressure produced by the first, aspiration vacuum pump. In some examples, the valve can reduce or eliminate forward pressure. This can prevent a clot from getting kicked off the distal tip of the aspiration catheter. In some embodiments, the use of the pulsatile pump can provide for rapid pulsing of vacuum pressure.

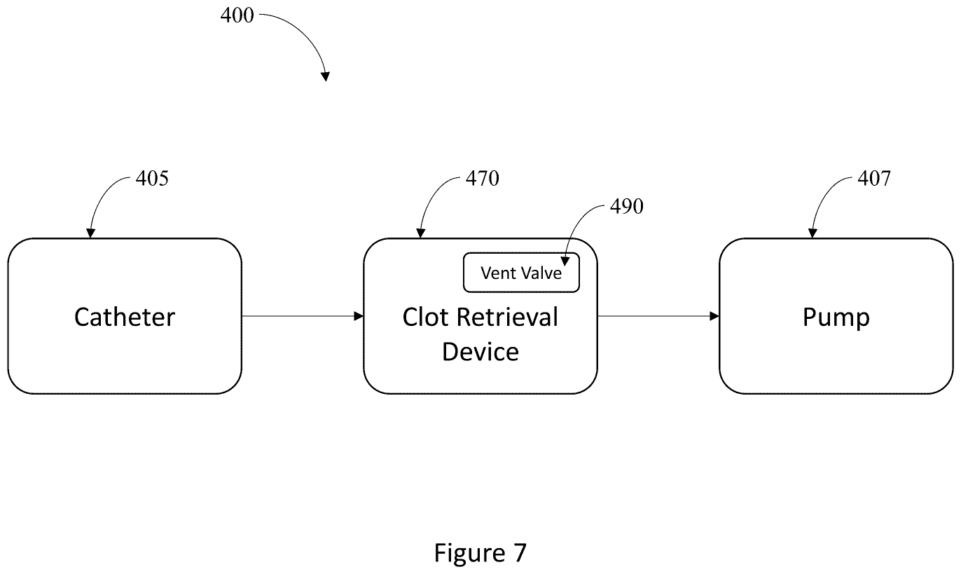

FIG. 7 illustrates another schematic of a fluid management system for aspiration procedures. The system 400 can include a catheter 405, a clot retrieval device 470, and a pump 407. As discussed above, the clot retrieval device 470 can include a valve 490 that is configured to create a pulse in the vacuum pressure. In some embodiments, the valve 490 can be manually engaged by the user. In some examples, the clot retrieval device 470 can include a sensor that allows the valve 490 to be automatically activated when the sensor detects that the catheter is blocked and no flow is present.

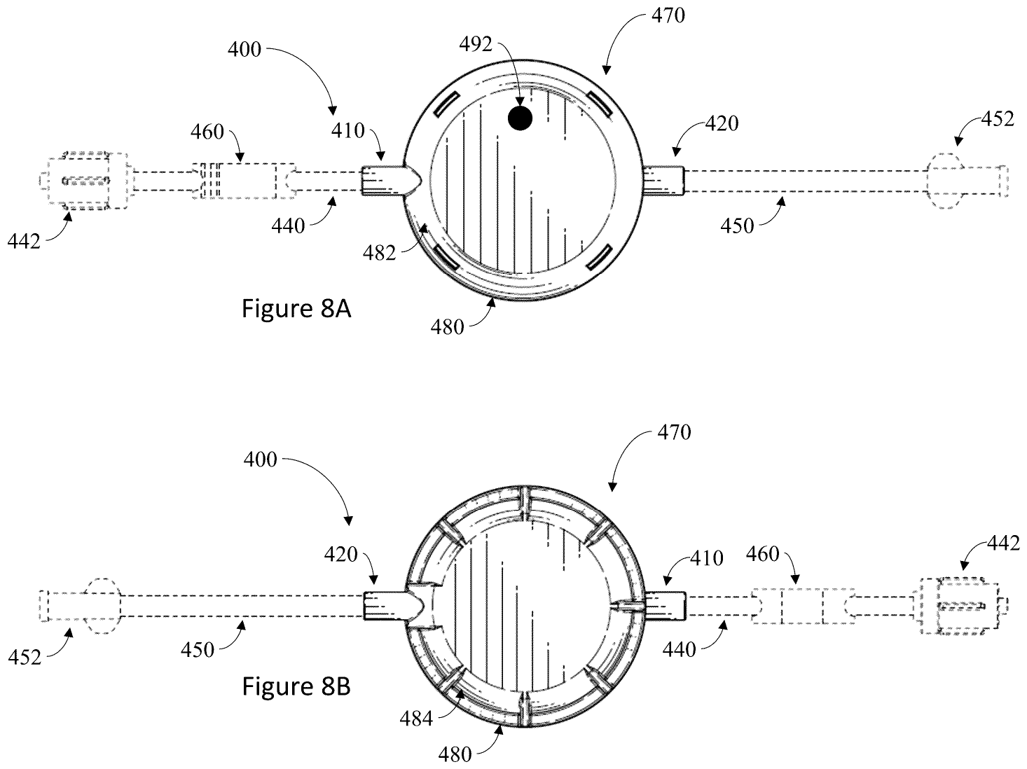

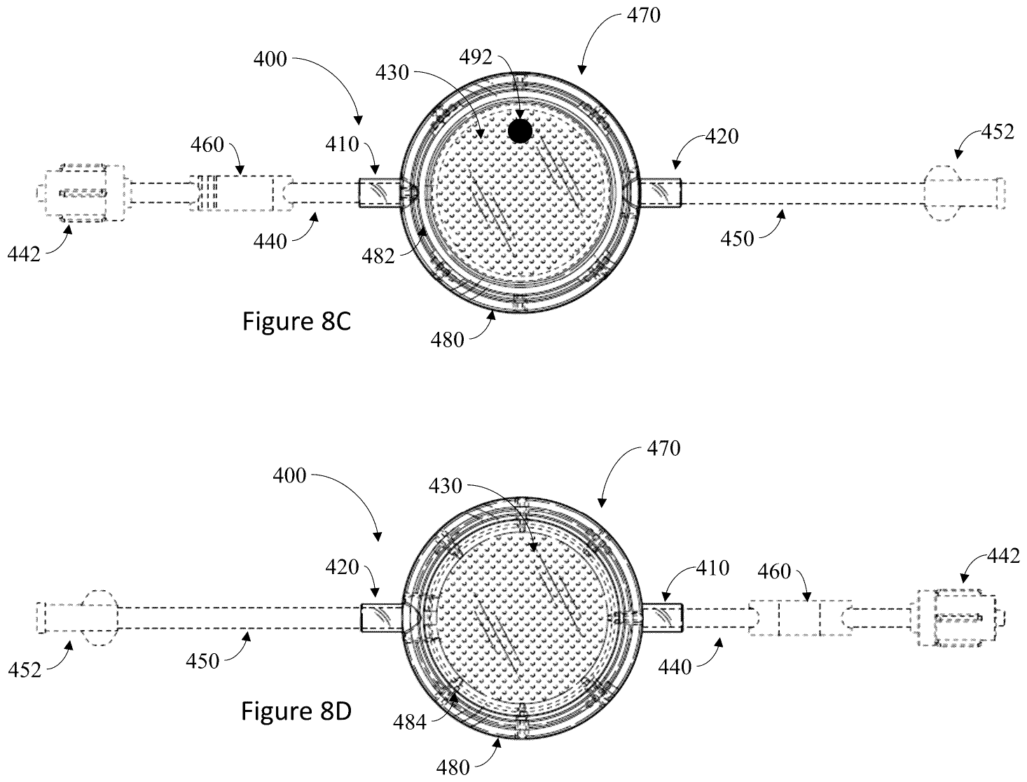

FIGS. 8A-8D illustrates an embodiment of a clot retrieval device 470 with a button 492 for actuating the valve 490. FIGS. 8A-8D resembles the system 300 in many respects. Accordingly, numerals used to identify components of the system 300 are incremented by a factor of one hundred to identify like features of the system 300. This numbering convention generally applies to the remainder of the figures. As mentioned above, any component or step disclosed in any embodiment in this specification can be used in other embodiments. As with the clot retrieval device 370, the clot retrieval device 470 can include a body 480 with a first catheter port 410 and a second catheter port 420. In some examples, the body 480 can include a third port—a flush port (not illustrated)—that is configured to allow the injection of saline or other fluid into the body 480 to improve clot visualization once it is trapped in the filter 430. In some embodiments, the body 480 includes a top portion 482 and a bottom portion 484. In some examples, the body 480 includes a filter 430 positioned between the top portion 482 and the bottom portion 484.

In some embodiments, the first catheter port 410 is configured to connect to a first end of a first catheter 440 that is fluidly connected to a proximal end of an aspiration catheter. In some examples, the first catheter 440 includes a connector 442 positioned at a second end of the first catheter 440 that is configured to engage or mate with a corresponding connector. In some embodiments, the second catheter port 420 is configured to connect to a first end of a second catheter 350 that is fluidly connected to an aspiration source (e.g., a pump). In some examples, the second catheter 350 includes a connector 452 positioned at a second end of the second catheter 350 that is configured to engage or mate with a corresponding connector. In some examples, the system 400 can include a clamp 460. The clamp 460 can be positioned over the first catheter 440 to allow the user to engage the clamp and provide flow control over the clot-retrieval device clot retrieval device 470.

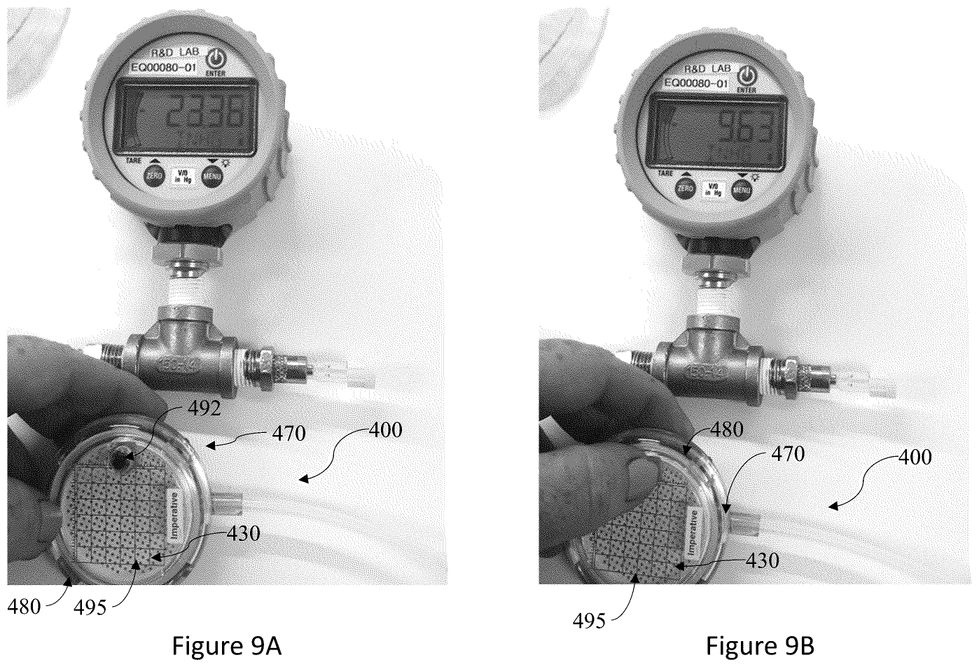

As illustrated in FIGS. 8A and 8C, the clot retrieval device 470 can include a button 492 for actuating a valve 490. In some examples, the button 492 can be positioned on the body 480 of the clot retrieval device 470. As shown in FIGS. 8A and 8C, in some embodiments, the button 492 is positioned on a top portion 482 of the body 480. As discussed above, although the valve 490 of the clot retrieval device 470 can be automatically actuated, in some examples, the clot retrieval device 470 can optionally provide for a button 492 that allows the user to actuate the valve 490. When the valve 490 is actuated, the valve 490 can release the vacuum and create a pulse in the vacuum pressure. This is illustrated in FIGS. 9A-9B that shows the pressure being applied to the system before and after the button 492 is actuated. As an example, the pressure applied to the system 400 before the button 492 is engaged is approximately −23.36 Hg. After the button 492 is actuated, the pressure applied to the system 400 is reduced to −9.63 Hg. As shown, the pressure applied to the system 400 is significantly lower once the button 492 is engaged (i.e., FIG. 9B). In some embodiments, because the valve 490 vents to a second vacuum source instead of to the air, there is not a decay in pressure and negative pressure is maintained (i.e., the pressure does not become positive).

In some embodiments, this can allow the user to provide rapid pulsing of vacuum pressure that can improve the ingestion of a clot during the clinical procedure. In some embodiments, the button 492 can also function as a clearing mechanism to allow the physician a way to visualize the clot midway through the procedure.

The clot retrieval device 470 can have a circular body with a circular cross-section. The clot retrieval device 470 can be made of a variety of materials such as plastics and polymers. In some embodiments, one or more components of the clot retrieval device 470 can be made of a transparent material, non-transparent material, partially transparent material, or any combinations thereof. As shown in FIGS. 8A-8D, in some examples, at least a portion of the clot retrieval device 470 can be transparent to improve clot visualization once it is trapped in the clot retrieval device 470. In some embodiment, at least one or both of the top portion 482 and the bottom portion 484 is transparent such that the filter 430 is visible through the body of the clot retrieval device 470.

As discussed above, the clot retrieval device 470 can include the filter 430. The filter 430 can be circular in shape. In some examples, the filter 430 can be secured between the top portion 482 and the bottom portion 484 of the body 480. In some embodiments, the clot retrieval device 470 may be provided with different pore sizes based on the needs of the physician or the parent. For example, in some examples, the filter 430 can include 1 mm holes that catches solid clot debris as it is driven through the mini canister. In some embodiments, the pore sizes may be at most 1 mm, at most 2 mm, at most 3 mm, at most 4 mm, at most 5 mm.

As discussed above, in some embodiments, the clot retrieval device can include a sensor. The sensor can determine when the catheter is blocked/corked (e.g., when flow is substantially reduced) to allow the valve 490 to be automatically actuated. This can be used to determine when pure aspiration versus cyclic aspiration is being triggered. In some examples, as long as flow is present, there would be no need to cycle the vacuum.

In some examples, the aspiration catheter 405 aspirates material which can flow through the second end of the first catheter 440 and enter the first catheter port 410 of the clot retrieval device 470. The aspirated material can then be filtered by the filter 430. Material that is larger than the openings on the filter 430 can be prevented from leaving the clot retrieval device 470, while material that is smaller than the openings on the filter 430 can flow out of the second catheter port 420 and the second catheter 450. In some embodiments, the user can slow down flow by engaging the clamp 460. In some examples, the user can engage the button 492 to engage the valve 490 and cause a pulsation in pressure in the system 400. By actuating the button 492, the negative pressure is reduced and by releasing the button 492, the negative pressure is increased. This pulsation in pressure can allow the aspiration catheter 405 to better aspirate a clot by “shaking” the clot loose. As well, actuating the button 492 can serve to perform a clearing mechanism such that the clot is better visualized within the body of the clot retrieval device 470. In some examples, the material retained by the clot retrieval device 470 can be removed from the clot retrieval device 470 and tested by the user.

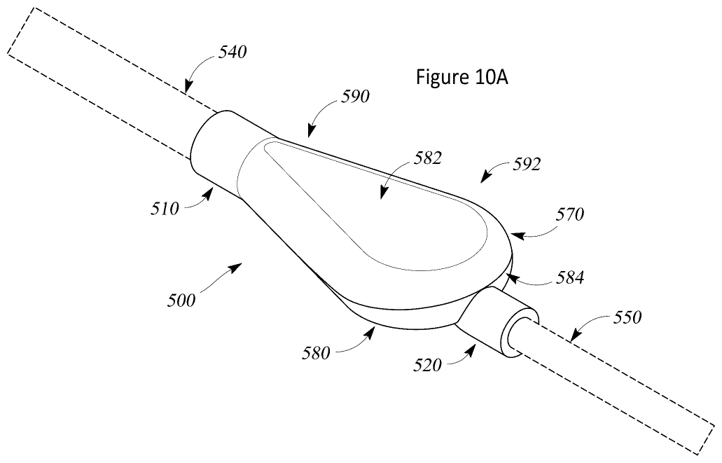

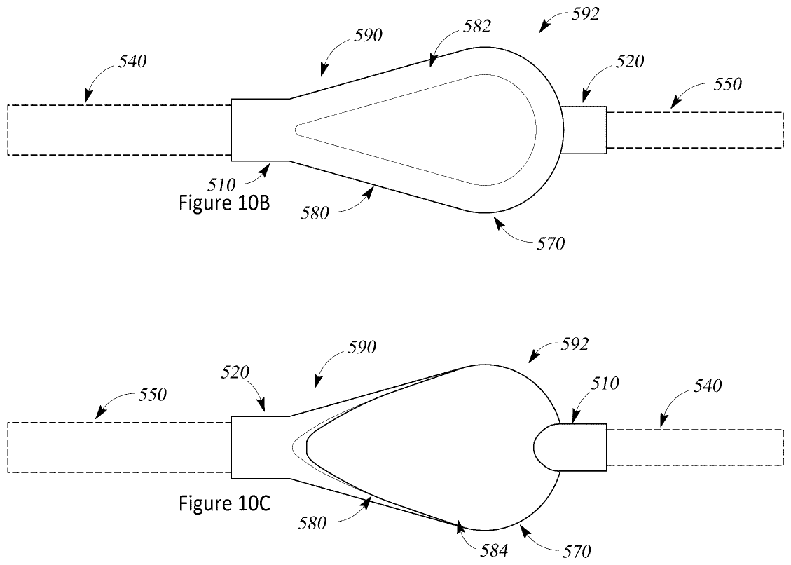

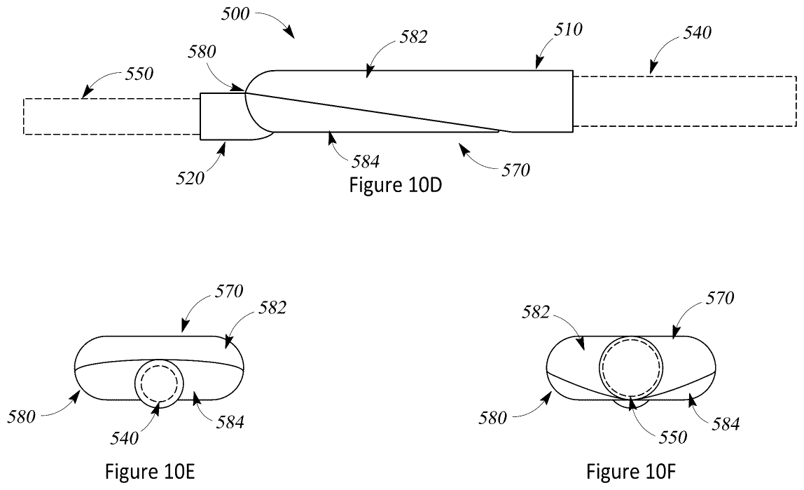

FIGS. 10A-10F illustrate various views of embodiments of a clot retrieval device 570. Unless otherwise noted, similar reference numerals in FIGS. 10A-10F refer to components that are the same as or generally similar to the components in the remaining figures discussed herein. For example, FIGS. 10A-10F may utilize reference numerals with the same last two digits as previous Figures and embodiments to reference components that are the same as or generally similar to components in previous Figures and embodiments, such as clot retrieval device 370 and clot retrieval device 570. As with all embodiments in this specification, it will be understood that any feature, structure, material, method, step, or component that is described and/or illustrated in the embodiments of FIGS. 10A-10E can be used with or instead of any feature, structure, material, method, step, or component that is described and/or illustrated in any other embodiment of this specification. It will also be understood that any feature, structure, material, method, step, or component of any embodiment described and/or illustrated herein can be used with or instead of any other feature, structure, material, method, step, or component of any embodiment of clot retrieval device 570 shown in FIGS. 10A-10F.

The broken lines in FIGS. 10A-10F denote portions of the clot retrieval device 570 that may not form part of a design. However, it is contemplated that lines that are currently illustrated as broken may be redrawn as solid lines and lines that are currently illustrated as solid may be redrawn as broken lines. The scope of the present disclosure encompasses all lines illustrated, whether broken or solid.

The clot retrieval device 570 may be made of a variety of materials such as plastics and polymers, in some embodiments, and one or more components of the clot retrieval device may be made of transparent material, non-transparent material, partially transparent material, or any combinations thereof.

FIGS. 10A-10F illustrate an embodiment of a system 500 including a clot retrieval device 570. The clot retrieval device 570 can include a body 580 enclosing a chamber which communicates with a first port 510 and a second port 520. In some embodiments, the body 580 includes a housing having a top portion 582 and a bottom portion 584. The body 580, in some instances, may be a unitary housing. In some examples, the body 580 includes a filter positioned in the chamber between the top portion 582 and the bottom portion 584.

In some examples, the first port 510 is configured to connect to a first end of a first tube 540 that is fluidly connected to a proximal end of an aspiration catheter. In some embodiments, the first tube 540 includes a connector positioned at a second end of the first tube 540 that is configured to engage or mate with a corresponding connector. In some embodiments, a second port 520 is configured to connect to a first end of a second tube 550 that is fluidly connected to an aspiration source (e.g., a pump). In some embodiments, the second tube 550 includes a connector positioned at a second end of the second tube 550 that is configured to engage or mate with a corresponding connector. In some examples, the system 500 can include a clamp.

The first side port 510 may be larger, smaller, or the same size as the second side port 520 and/or may be configured to fluidly connect to tubes that are larger, smaller, or the same size as the second side port 520. In some instances, the first side port 510 may be larger than the second side port 520 such that the first side port 510 is configured to fluidly connect to a tube (e.g., first tube 540) being larger than a second tube (e.g., second tube 550) that may be fluidly connected to the second side port 520. The first side port 510, in some embodiments, may be configured to fluidly connect to a standard tube (e.g., a tube having an inner diameter of about 0.1 inches). The second side port 520 may be smaller than the first side port 510 such that second tube 550 is smaller than first tube 540. In some instances, the smaller size of the second tube 550 may advantageously facilitate in decreasing a priming time of the system such that the aspiration source is configured to “prime” (e.g., apply negative pressure at least throughout the length of the second tube and the clot retrieval device 570). The smaller tube size may facilitate priming by decreasing the overall internal tube volume between the aspiration source and the clot retrieval device 570 that requires priming. In some instances, the smaller tube size may advantageously decrease a blood fluid flow rate through the second tube 550 so as to also reduce a potential amount of blood loss during an aspiration procedure.

The body 580 of the clot retrieval device 570 may be formed with a size and/or shape to promote fluid flow through the body 580 between the first port 510 and the second port 520. In some instances, the body 580 may be configured to inhibit the formation of blood “pooling” within the clot retrieval device 570 such that, as blood is flowing through the clot retrieval device 570, the chamber of the body 580 does not accumulate stagnant or low-flowing blood in areas within the chamber. For example, the body 580 may be configured to facilitate complete drainage of the chamber of the body 580.

In some instances, the body 580 may have a first body portion 590 located proximate to the first port 510 at a first end of the body 580 that comprises a width smaller than a second body portion 592 locate proximate to the second port 520 at a second end of the body 580. The body 580 may taper between the smaller first body portion 590 and the larger second body portion 592 to form a “tear-drop” or funnel-like configuration. This configuration may advantageously inhibit the occurrence of blood pooling within the chamber of the body 580 during use. In some instances, this configuration may advantageously facilitate fluid drainage of the chamber. The design, in some embodiments, may also decrease a weight and/or overall size of the device to facilitate use.

Any embodiment of a clot retrieval device as discussed in this specification and/or as illustrated in the Figures may further include a measurement component to facilitate determining a size and/or volume of a clot capture within the chamber of the body during use. In some instances, at least a portion of the body (e.g., any surface along a top portion or bottom portion) may include a measurement guide that permits a user to readily determine a size and/or volume of a clot capture within the chamber. For example, as illustrated in FIGS. 9A and 9B, a transparent portion of the body may include a grid, scale, or any suitable marking 495 to visually permit a user to determine a size of the clot. It will be understood that this may be utilized in any embodiment of a clot retrieval device as described herein.

In some instances, the body of the clot retrieval device may be sized and/or shaped to measure a volume of the clot during the procedure. For example, the clot retrieval device can include an elongated tubular body (e.g., a cylindrical body) that may cause the clot to elongate within the chamber of the tubular body as the clot passes along a filter. Elongation of the clot along an internal surface of the tubular body may advantageously facilitate measurement of the volume of the clot relative to the length of the clot.

Any embodiment of a clot retrieval device as discussed in this specification and/or as illustrated in the Figures may further include a coating along at least a portion of an interior surface and/or of a filter of the clot retrieval device in the body chamber to provide one or more of a variety of properties to the clot retrieval device. In some instances, the coating may be configured to enhance visualization through at least a portion of the body of the clot retrieval device. The coating may be configured to inhibit blood accumulation or increase blood repellant properties. In some instances, the clot retrieval device may comprise a coating to inhibit foam formation during an aspiration procedure. The coatings may be located at least partially along an interior surface of the body. The coating can be both hydrophobic and oleophobic. In some instances, the coating may have some hydrophilic features on a portion of the polymer to increase olcophobic properties.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention. The drawings are for the purpose of illustrating embodiments of the invention only, and not for the purpose of limiting it.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “deploying an instrument sterilized using the systems herein” include “instructing the deployment of an instrument sterilized using the systems herein.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

Any titles or subheadings used herein are for organization purposes and should not be used to limit the scope of embodiments disclosed herein.

### Example Embodiments

A clot filtering device comprising one or more of the following:
a body comprising:
   a first body portion;
   a second body portion being configured to form an internal cavity with the first body portion;
   a first port being located on a first side of the body;
   a second port being located on a second side of the body, the second port being in fluid communication with the first port through the internal cavity, the second port being configured to be placed in fluid communication with a first vacuum source; and
   a valve being configured to selectively permit fluid communication with at least one of atmosphere or a second vacuum source; and
a filter being located within the internal cavity of the body in between the first port and the second port, the filter comprising a plurality of openings.

A clot filtering device comprising one or more of the following:
a body comprising:
   a first body portion;
   a second body portion being configured to form an internal cavity with the first body portion;
   a first port being located on a first side of the body; and
   a second port being located on a second side of the body, the second port being in fluid communication with the first port through the internal cavity,
   wherein at least one of the first body portion and the second body portion is transparent to permit visualization of the internal cavity; and
a filter being located within the internal cavity of the body in between the first port and the second port, the filter comprising:
   a plurality of openings; and
   a first side residing on a plane positioned at a nonorthogonal angle relative to a longitudinal axis of the first port.

A clot filtering device comprising one or more of the following:
a body comprising:
   a first body portion;
   a second body portion being configured to form an internal cavity with the first body portion;
   a first port being located on a first side of the body;
   a second port being located on a second side of the body, the second port being in fluid communication with the first port through the internal cavity; and
   a third port being configured to permit injection of fluid into the internal cavity; and
   a filter being located within the internal cavity of the body in between the first port and the second port, the filter comprising a plurality of openings.

A clot filtering device comprising:
a body comprising:
   a first body portion;
   a second body portion being configured to form an internal cavity with the first body portion;
   a first port being located on a first side of the body; and
   a second port being located on a second side of the body, the second port being in fluid communication with the first port through the internal cavity,
   wherein the internal cavity has:
      a depth defined as a first distance being transverse to a longitudinal axis of the body and being between the first body portion and the second body portion, and
      a width defined as a second distance transverse to the depth and transverse to the longitudinal axis of the body, the width being smaller than the depth; and
a filter being located within the internal cavity of the body in between the first port and the second port, the filter comprising a plurality of openings.

The clot filtering device of any embodiment described herein, wherein the body comprises a tubular sidewall.

The clot filtering device of any embodiment described herein, wherein at least one side of the filter resides on a plane positioned at a nonorthogonal angle relative to a longitudinal axis of the body.

The clot filtering device of any embodiment described herein, wherein the nonorthogonal angle is between 30 to 90 degrees relative to the longitudinal axis of the body.

The clot filtering device of any embodiment described herein, wherein each of the plurality of openings is less than or equal to 1 mm.

The clot filtering device of any embodiment described herein, wherein the plurality of openings are configured to inhibit a clot from passing through the filter.

The clot filtering device of any embodiment described herein, wherein the first port and the second port are positioned on opposite ends of the body.

The clot filtering device of any embodiment described herein, wherein the body further comprises a third port being configured to permit injection of fluid into the internal cavity.

The clot filtering device of any embodiment described herein, wherein at least one of the first body portion or the second body portion is at least partially transparent to permit visualization of the internal cavity.

The clot filtering device of any embodiment described herein, wherein the body further comprises a button configurated to permit a user to manually actuate the valve.

The clot filtering device of any embodiment described herein, wherein the button is positioned on a top portion of the body.

The clot filtering device of any embodiment described herein, further comprising a sensor being configured determine when fluid flow through body has stopped.

The clot filtering device of any embodiment described herein, wherein the valve is automatically engaged when the sensor has determined that fluid flow has stopped.

What is claimed is:

1. An aspiration system comprising:
an aspiration pump configured for location outside of a sterile field;
a chamber configured for location within the sterile field;
a first tube having a first inner diameter, the first tube configured to provide a fluid flow path for aspirated material into the chamber; and
a second tube having a second inner diameter, the second tube configured to extend between the chamber and the aspiration pump and configured to place the chamber and the aspiration pump in communication with each other;
wherein the first inner diameter is larger than the second inner diameter.

2. The aspiration system of claim 1, wherein the first tube is configured to fluidly couple the chamber with a distal end of an aspiration catheter.

3. The aspiration system of claim 2, wherein the chamber is configured to capture a clot aspirated by the aspiration catheter.

4. The aspiration system of claim 1, further comprising an aspiration catheter configured to be in communication with the first tube, wherein the first tube and the aspiration catheter are separate components.

5. The aspiration system of claim 4, wherein the first tube is fluidly coupled to a proximal end of the aspiration catheter.

6. The aspiration system of claim 1, wherein the chamber is enclosed within a body comprising a first port and a second port, the first port fluidly connected to the first tube, and the second port fluidly connected to the second tube.

7. The aspiration system of claim 5, wherein a size of the first port is larger than a size of the second port.

8. The aspiration system of claim 5, wherein the body further comprises a third port configured to permit injection of fluid into the chamber.

9. The aspiration system of claim 5, wherein the chamber is in fluid communication with the first port and the second port.

10. The aspiration system of claim 5, wherein the first port and the second port are positioned on opposite ends of the body.

11. The aspiration system of claim 1, wherein the chamber comprises a filter.

12. The aspiration system of claim 1, further comprising a valve configured to selectively permit fluid communication with at least one of atmosphere or the aspiration pump, and a button configurated to permit a user to manually actuate the valve.

13. The aspiration system of claim 1, further comprising a sensor configured to determine when a fluid flow through the chamber has stopped.
